(19) United States
(12) Patent Application Publication
(10) Pub. No.: US 2025/0275689 A1
(43) Pub. Date: Sep. 4, 2025

(54) SYSTEM AND METHOD FOR PREDICTING THAT AN INDIVIDUAL WILL FALL

(71) Applicant: KARETEQ, Colomiers (FR)

(72) Inventors: Karim Ben Dhia, Colomiers (FR); Thomas Carrie, Toulouse (FR); Nadim Nasreddine, Toulouse (FR)

(73) Assignee: KARETEQ, Colomiers (FR)

(21) Appl. No.: 18/692,797

(22) PCT Filed: Sep. 16, 2022
(86) PCT No.: PCT/EP2022/075747

§ 371 Date: Aug. 10, 2024

(30) Foreign Application Priority Data
Sep. 16, 2021 (FR) FR2109756
Sep. 15, 2022 (FR) FR2209307

(51) Int. Cl. A61B 5/11; A61B 5/00; G08B 21/04; G08B 31/00; G16H 40/67; G16H 50/70

(52) U.S. Cl. CPC A61B 5/1117; A61B 5/7275; G08B 21/043; G08B 21/0446; G08B 31/00; G16H 40/67; G16H 50/70

(57) ABSTRACT

The invention relates to a system and a method for predicting that an individual will fall, comprising: a casing (10) worn by an individual (8), in which casing there is housed a plurality of sensors for acquiring measurements representative of the posture and/or movements of the individual; a unit (100) for processing the measurements provided by said plurality of sensors and comprising: a module (110) for determining a profile score, based on data representative of characteristics specific to the individual; a module (120) for computing motive indices, based on the measurements provided by said plurality of sensors and representative of the activity of the individual over a predetermined time interval; a module (130) for computing a fall risk score, based on said motive indices and on said profile score; a module (140) for determining a risk of falling, based on a variation in said fall risk score beyond a predetermined threshold over a predetermined time interval.

### TECHNICAL FIELD OF THE INVENTION

The invention relates to a system and a method for predicting that an individual will fall, i.e. a system and a method configured to evaluate and anticipate the risk that an individual will fall. The invention also relates to a system and a method for detecting that an individual has fallen, implementing a system and a method for fall prediction in accordance with the invention.

### TECHNOLOGICAL BACKGROUND

Nowadays, there are numerous smart devices intended to detect that people likely to fall have fallen, such as elderly people, people suffering from attention deficit disorder, people liable to have panic attacks, people with epilepsy, etc.

These devices and methods generally use movement sensors (such as accelerometers) worn by the people to be monitored, which are associated with units for processing the data provided by these sensors. The processing unit analyses and interprets the values of the data provided by the sensors and triggers a fall detection alert as soon as the values exceed a predetermined threshold. The proposed processing consists for example of counting the number of steps made by the monitored person over a predetermined time period and of triggering the alert if no activity is observed over a normal period of activities.

These devices are available nowadays in the form of pedometers, smart watches, pendants, smartphones, etc. which are carried by the individuals to be monitored. The processing units can be incorporated in the devices or be housed on remote servers connected to the detection devices by wireless connection means.

These solutions are useful to be able to react rapidly once the fall is detected by the system, which makes it possible to make contact quickly with the monitored person and/or send help quickly so that the person can be taken care of.

That being said, these systems cannot prevent the fall. In other words, the known solutions only allow the fall to be detected and the time for intervention between the fall and care arriving for the individual who has fallen to be reduced.

The inventors have sought to develop this and provide a system which makes it possible to predict the fall before it has occurred so as to be able to prevent it. In order words, the inventors have sought to develop a system and a method for predicting that an individual will fall, i.e. a system and a method which make it possible to detect that a monitored individual is imminently about to fall so as to be able to alert the individual (or a care giver) that there is a risk of falling and thereby prevent the fall occurring or reduce the consequences thereof.

### AIMS OF THE INVENTION

The invention aims to provide a system and a method for predicting a risk that an individual will fall.

The invention also aims to provide, in at least one embodiment, a system and a method which are adapted to the physical and/or physiological characteristics of the monitored person.

The invention also aims to provide, in at least one embodiment, a system and a method which can interact with the monitored individual to be able to alert said individual (or a care giver) of a risk of falling and/or confirm a possible detected fall.

The invention also aims to provide, in at least one embodiment, a system and a method which make it possible to monitor activity parameters of a monitored individual.

The invention also aims to provide a system and a method for detecting that an individual has fallen, which implements a system and a method for predicting a risk that an individual will fall in accordance with the invention.

### DESCRIPTION OF THE INVENTION

In order to do this, the invention relates to a system for predicting that an individual will fall, comprising:
a casing configured to be able to be worn by an individual, said casing comprising a plurality of sensors for acquiring measurements representative of the posture and/or movements of the individual, including at least one accelerometer, magnetometer and gyroscope,
a unit for processing the measurements provided by said plurality of sensors.

The system in accordance with the invention is characterized in that said processing unit comprises at least:
a module for determining an item of data, referred to as profile score, based on data representative of characteristics specific to the individual,
a module for computing data, referred to as motive indices, based on the measurements provided by said plurality of sensors and representative of the activity of the individual over a predetermined time interval,
a module for computing an item of data, referred to as fall risk score, based on said motive indices and on said profile score,
a module for determining a risk of falling, based on a variation in said fall risk score beyond a predetermined threshold over a predetermined time interval.

The system in accordance with the invention thus has the feature of processing data acquired by sensors housed in a casing worn by the monitored individual to deduce therefrom a risk of falling from the variation in a risk score beyond a predetermined threshold. The fall risk score is derived from motive indices and a profile score specific to the individual. The motive indices reflect the activity of the monitored person and provide a representation of the physiological factors of the monitored person.

In other words, the system in accordance with the invention continuously monitors the fall risk score and triggers an alert as soon as this risk score varies rapidly over a predetermined time period, which is characteristic of a severe deterioration in the stability of the monitored person.

Throughout the text, the phrases “monitored person”, “person to be monitored”, “monitored individual” or “individual to be monitored” refer to the person liable to fall who uses the system in accordance with the invention.

The prediction of the fall is specific to each person in the sense that it is based on a profile score which depends on the characteristics specific to the monitored individual, and motive indices which are based on the activity of the person. Therefore, and in contrast to the majority of the known systems, the invention adapts to the specificities of the person.

Advantageously and in accordance with the invention, said module for determining said profile score of said individual comprises an automatic computing model trained to determine a profile score, this computing model, referred to as first model, having been trained using a training database, referred to as profile bank, which comprises data representative of specific characteristics of a plurality of individuals associated with detected fall occurrences for said plurality of individuals.

According to this advantageous variant, the determination of the profile score of an individual is based on an automatic computing model trained using a profile bank. This profile bank is formed of the specific characteristics of a plurality of individuals associated with detected fall occurrences for this plurality of individuals.

The use of such an artificial intelligence model makes it possible to automatically detect patterns which are characteristic of a risk of falling.

The automatic computing model implemented by the module for determining the profile score of an individual can be of any type. It can be a supervised learning neural network, a support vector machine (SVM) or any other machine learning algorithm.

This module thus makes it possible to attribute a profile score to each individual using the system in accordance with the invention, said score depending on the characteristics specific to the individual.

Advantageously and in accordance with the invention, the data representative of the characteristics specific to each individual comprise one or more items of information related to the age, sex, prescribed medications, weight, size, sight, hearing, fall history, etc. of said individual.

According to this advantageous variant, the characteristics specific to the individual used to determine a profile score are items of information related to the age, sex, prescribed medications, weight, size, sight, hearing, fall history, etc. thereof.

A weighting specific to each characteristic can be used. For example, the age of the individual can be assigned a weighting of 90%, the sex can be assigned a weighting of 70%, the number of prescribed daily medications can be assigned a weighting of 75%, wearing glasses can be assigned a weighting of 50%, fall history can be assigned a weighting of 100%, the weight of the individual can be assigned a weighting of 50% and the size of the individual can be assigned a weighting of 25%. This weighting characterizes the importance of the criterion in the determination of the profile score.

Of course, the indicated weighting values are only one example of the importance given to the different criteria in the determination of the profile score and a different assignment of the weighting to each criterion can be made without compromising the core concept of the invention.

Once the profile score of the individual is known, the system in accordance with the invention can compute the motive indices of the individual over time. These motive indices aim to represent the activity and behavior of the individual over predetermined time periods.

Advantageously and in accordance with the invention, said motive indices computed by said computing module are selected from the group comprising:
indices representative of the average activity of the individual over a predetermined time interval T, referred to as SMA indices, computed from the measurements provided by each of the sensors housed in said casing in accordance with the following equations:

$$S M {A}_{A} = \frac{1}{T} ( {\int }_{t}^{t + T} \vert {a}_{t}^{x} \vert dt + {\int }_{t}^{t + T} \vert {a}_{t}^{y} \vert dt + {\int }_{t}^{t + T} \vert {a}_{t}^{z} \vert dt ) \qquad [ Equation 1 ]$$

where aₜ^{x}, aₜ^{y}, aₜ^{z} represent the acceleration values measured on three axes of a direct trihedron (x, y, z) of said accelerometer,

$$S M {A}_{M} = \frac{1}{T} ( {\int }_{t}^{t + T} \vert {m}_{t}^{x} \vert dt + {\int }_{t}^{t + T} \vert {m}_{t}^{y} \vert dt + {\int }_{t}^{t + T} \vert {m}_{t}^{z} \vert dt ) \qquad [ Equation 2 ]$$

where mₜ^{x}, mₜ^{y}, mₜ^{z} represent the magnetic values measured on three axes of said direct trihedron (x, y, z) of the magnetometer,

$$S M {A}_{G} = \frac{1}{T} ( {\int }_{t}^{t + T} \vert {g}_{t}^{x} \vert dt + {\int }_{t}^{t + T} \vert {g}_{t}^{y} \vert dt + {\int }_{t}^{t + T} \vert {g}_{t}^{z} \vert dt ) \qquad [ Equation 3 ]$$

where gₜ^{x}, gₜ^{y}, gₜ^{z} represent the gyroscopic values measured on three axes of said direct trihedron (x, y, z) of the gyroscope,
indices representative of the current activity of the individual computed from the measurements provided by each of the sensors housed in said casing in accordance with the following equations:

$${‖ A ‖}_{t} = \sqrt{{a}_{t}^{{x}^{2}} + {a}_{t}^{{y}^{2}} + {a}_{t}^{{z}^{2}}} , \qquad [ Equation 4 ]$$

$${‖ M ‖}_{t} = \sqrt{{m}_{t}^{{x}^{2}} + {m}_{t}^{{y}^{2}} + {m}_{t}^{{z}^{2}}} \qquad [ Equation 5 ]$$

$${‖ G ‖}_{t} = \sqrt{{g}_{t}^{{x}^{2}} + {g}_{t}^{{y}^{2}} + {g}_{t}^{{z}^{2}}} \qquad [ Equation 6 ]$$

indices representative of the energy of said individual, referred to as HA indices, computed as the variation in the current activity over said predetermined period T in accordance with the following equations:

$$H {A}_{T}^{A} = var ( {‖ A ‖}_{t} ) \qquad [ Equation 7 ]$$

where var represents the variation in the value over the predetermined time interval T,

$$H {A}_{T}^{M} = var ( {‖ M ‖}_{t} ) \qquad [ Equation 8 ]$$

where var represents the variation in the value over the predetermined time interval T,

$${HA}_{T}^{G} = va r ( ‖ G {‖}_{t} ) \qquad [ Equation 9 ]$$

where var represents the variation in the value over the predetermined time interval T,
indices representative of the harmony of the activity of the individual, referred to as HM indices, over said predetermined time interval in accordance with the following equations:

$${HM}_{T}^{A} = \sqrt{\frac{va r ( {‖ A ′ ‖}_{t} )}{va r ( {‖ A ‖}_{t} )}} where {‖ {A}^{′} ‖}_{t} = \frac{Δ ‖ A {‖}_{t}}{Δ t} \qquad [ Equation 10 ]$$

$${HM}_{T}^{M} = \sqrt{\frac{va r ( {‖ M ′ ‖}_{t} )}{va r ( {‖ M ‖}_{t} )}} where {‖ {M}^{′} ‖}_{t} = \frac{Δ ‖ M {‖}_{t}}{Δ t} \qquad [ Equation 11 ]$$

$${HM}_{T}^{G} = \sqrt{\frac{va r ( {‖ G ′ ‖}_{t} )}{va r ( {‖ G ‖}_{t} )}} where {‖ {G}^{′} ‖}_{t} = \frac{Δ ‖ G {‖}_{t}}{Δ t} \qquad [ Equation 12 ]$$

indices representative of the irregularities in the frequency range during the activities of the individual over said predetermined time interval T, referred to as HC indices, measured in accordance with the following equations:

$${HC}_{T}^{A} = \sqrt{\frac{va r ( {‖ A ″ ‖}_{t} )}{va r ( {‖ A ‖}_{t} )}} where {‖ {A}^{″} ‖}_{t} = \frac{Δ ‖ A ′ {‖}_{t}}{Δ t} \qquad [ Equation 13 ]$$

$${HC}_{T}^{M} = \sqrt{\frac{va r ( {‖ M ″ ‖}_{t} )}{va r ( {‖ M ‖}_{t} )}} where {‖ {M}^{″} ‖}_{t} = \frac{Δ ‖ M ′ {‖}_{t}}{Δ t} \qquad [ Equation 14 ]$$

$${HC}_{T}^{G} = \sqrt{\frac{va r ( {‖ G ″ ‖}_{t} )}{va r ( {‖ G ‖}_{t} )}} where {‖ {G}^{″} ‖}_{t} = \frac{Δ ‖ G ′ {‖}_{t}}{Δ t} \qquad [ Equation 15 ]$$

The system according to this variant makes it possible to compute a certain number of motive indices representative of the activity of the monitored individual.

In particular, the SMA indices make it possible to characterize the average activity of the monitored individual. Computing these indices over predetermined periods reflects the individual's dynamics and his/her average activity over a day for example. These SMA indices are measurements of the sedentarity of the monitored individual and are computed on the accelerations (measurements provided by the accelerometer), the magnetic field (measurements provided by the magnetometer) and angular velocities (measurements provided by the gyroscope).

The HA indices make it possible to characterize the energy of the monitored individual. The computing of these indices over predetermined periods reflects the walking speed of the individual. These HA indices are computed on the accelerations (measurements provided by the accelerometer), the magnetic field (measurements provided by the magnetometer) and angular velocities (measurements provided by the gyroscope).

The HM indices make it possible to characterize the harmony in the activities of the monitored individual. The computing of these indices over predetermined periods reflects the walking symmetry and stability of the individual. These HM indices are computed on the accelerations (measurements provided by the accelerometer), the magnetic field (measurements provided by the magnetometer) and angular velocities (measurements provided by the gyroscope).

The HC indices make it possible to characterize the irregularities in the frequency range during the activities of the monitored individual. The computing of these indices over predetermined periods reflects the walking balance and stability of the monitored individual. These HC indices are computed on the accelerations (measurements provided by the accelerometer), the magnetic field (measurements provided by the magnetometer) and angular velocities (measurements provided by the gyroscope).

Advantageously and in accordance with the invention, said module for determining said fall risk score comprises an automatic computing model trained to determine a fall risk score, this computing model, referred to as second model, having been trained using a training database, referred to as risk score bank, which comprises values of said motive indices and profile scores of a plurality of individuals associated with detected fall occurrences for said plurality of individuals.

According to this advantageous variant, the determination of the fall risk score of an individual is based on an automatic computing model trained using a learning database (referred to as risk score bank). This learning database is formed of the motive indices and profile scores of a plurality of individuals likely to fall associated with detected fall occurrences for this plurality of individuals. This learning database is preferably the profile bank enhanced with the motive indices.

In order to build such a learning database, a plurality of individuals were provided with a casing of the system in accordance with the invention in order to be able to regularly compute the motive indices. These individuals were then observed (by a supervisor or using a dedicated device) in order to determine the fall occurrences. The learning database thus made it possible to associate values of the motive indices, computed prior to the occurrence of the fall, and the profile score of the individual with each fall occurrence. Once this first learning database is built, the system according to this variant can be used to detect the fall occurrence and thus improve and enhance the learning database.

The use of such an artificial intelligence model makes it possible to automatically detect patterns which are characteristic of a risk of falling.

The automatic computing model implemented by the module for determining the fall risk score of an individual can be of any type. It can be a supervised learning neural network, a support vector machine (SVM) or any other machine learning algorithm.

This module thus makes it possible to attribute a fall risk score to each individual using the system in accordance with the invention, said score depending on the value of the computed motive indices and the characteristics specific to the individual.

Advantageously and in accordance with the invention, said module for computing said risk of falling comprises an automatic computing model trained to determine a risk of falling, this computing model, referred to as third model, having been trained using a training database, referred to as risk bank, which comprises data representative of variations in the fall risk scores of a plurality of individuals associated with detected fall occurrences for said plurality of individuals.

According to this advantageous variant, the determination of the risk of falling of an individual is based on an automatic computing model trained using a learning database (referred to as risk bank). This learning database is formed of the motive indices and fall risk scores of a plurality of individuals likely to fall associated with detected fall occurrences for this plurality of individuals. This learning database is preferably the profile bank enhanced with the motive indices and the fall risk scores.

The use of such an artificial intelligence model makes it possible to automatically detect patterns which are characteristic of a risk of falling.

The automatic computing model implemented by the module for determining the fall risk score of an individual can be of any type. It can be a supervised learning neural network, a support vector machine (SVM) or any other machine learning algorithm.

This module thus makes it possible to attribute a risk of falling to each individual using the system in accordance with the invention, said risk of falling depending on the value of the computed motive indices, the fall risk score of the individual and the characteristics specific to the individual.

The different automatic computing models implemented by these advantageous variants of the invention can be enhanced with the results provided by the system and by the users themselves by confirming or discounting the fall risk alerts triggered by the system.

Advantageously and in accordance with the invention, the system further comprises a radio module configured to be able to transmit the data determined and computed by said processing unit to a remote server.

According to this aspect of the invention, the processing unit can transmit, to a remote server, the results of the processing and in particular the profile score, the fall risk score, the motive indices and the risk of falling which are computed by the different modules of the processing unit. The radio module also makes it possible to configure the system when it is used for the first time and to verify the presence of the system. The remote server can be used to refine the processing performed by the processing unit or to partly effect the processing. In this regard, the processing unit can be partially or completely housed in the item worn by the monitored individual or be partially or completely formed by a remote server. In the case where the processing unit is completely or partially formed by a remote server, the radio module forms wireless communication means configured to transmit the data coming from the sensors to said processing unit.

Advantageously and in accordance with the invention, the system further comprises an audio module comprising a microphone and a loudspeaker configured to permit an exchange of voice information between the individual and a remote operator.

According to this aspect of the invention, the audio module makes it possible to transmit an audio alert to the individual, for example when a risk of falling has been detected by the system. This radio module can likewise make it possible to receive voice information from the user, for example to discount a risk of falling or to confirm that the alert has indeed been received. Other uses can be envisaged depending upon the applications being targeted.

Advantageously and in accordance with the invention, the system further comprises a man-machine interface configured such that said individual can interact with said system and/or a remote operator and receive fall risk notifications.

According to this aspect of the invention, the man-machine interface makes it possible to display messages intended for the monitored individual (visual notifications). This man-machine interface can also comprise emergency buttons which the monitored person can press when he/she falls or requires aid.

The invention also relates to a method for predicting that an individual will fall, comprising:
acquiring measurements representative of the posture and/or movement of the individual from at least one accelerometer, magnetometer and gyroscope,
processing the acquired measurements.

The method in accordance with the invention is characterized in that it further comprises:
determining an item of data, referred to as profile score, based on data representative of characteristics specific to the individual,
computing data, referred to as motive indices, based on the acquired measurements and representative of the activity of the individual over a predetermined time interval,
computing an item of data, referred to as fall risk score, based on said motive indices and on said profile score,
determining a risk of falling, based on a variation in said fall risk score beyond a predetermined threshold over a predetermined time interval. The technical effects and advantages of the system in accordance with the invention apply mutatis mutandis to a method in accordance with the invention.

The invention also relates to a system for detecting that an individual has fallen, comprising:
a fall detection module configured to detect that said individual has fallen from at least one measurement from at least one sensor worn by said individual which exceeds a predetermined threshold,
a fall prediction system in accordance with the invention configured to determine a fall risk score for said individual,
a module for modifying said predetermined threshold of said fall detection module based on said fall risk score provided by said fall prediction system.

A fall detection system in accordance with the invention thus makes it possible to adjust the fall detection threshold based on the fall risk score provided by the fall prediction system in accordance with the invention. Therefore, it is possible to adapt the fall detection system to the individual. Therefore, the sensitivity of the fall detection system and its alert policy can evolve and be automatically adapted to the monitored individual. Furthermore, such a system can predict the fall before it occurs by integrating the fall prediction system in accordance with the invention.

The invention also relates to a fall prediction system, a fall detection system and a method, which are characterized in combination by all or some of the features mentioned above or below.

### LIST OF FIGURES

Other aims, features and advantages of the invention will become apparent upon reading the following description given solely in a non-limiting way and which makes reference to the attached figures in which:

FIG. 1 is a schematic view of a system in accordance with one embodiment of the invention,

FIG. 2 is a more detailed schematic view of a casing of a system in accordance with one embodiment of the invention,

FIG. 3 is a schematic block diagram of a processing unit of a system in accordance with one embodiment of the invention.

### DETAILED DESCRIPTION OF AN EMBODIMENT OF THE INVENTION

In the figures, for the purposes of illustration and clarity, scales and proportions have not been strictly respected. Furthermore, identical, similar or analogous elements are designated by the same reference signs in all the figures.

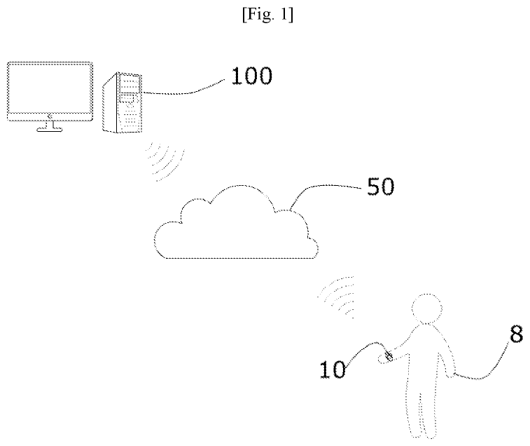

FIG. 1 illustrates a system in accordance with one embodiment of the invention comprising a casing 10 worn by an individual 8 to be monitored and a unit 100 for processing data acquired by sensors housed in the casing 10.

In FIG. 1, the casing 10 is integrated in a watch worn by the monitored individual. In accordance with other embodiments, the casing can be integrated in a pendant, in a pair of glasses or any item worn by the individual to be monitored.

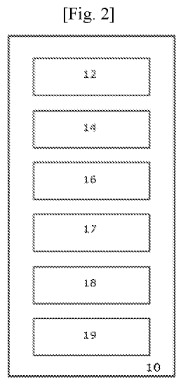

As shown in FIG. 2, this casing 10 houses sensors for acquiring measurements representative of the posture and movements of the individual. These sensors comprise at least one accelerometer 12, magnetometer 14 and gyroscope 16.

The accelerometer 12 is preferably an accelerometer with three axes so as to be able to directly obtain acceleration measurements on the three axes of a direct trihedron (x, y, z). In accordance with another embodiment, three single-axis accelerometers can be used which are oriented with respect to each other according to the above-mentioned trihedron. It is also possible to use a single single-axis accelerometer, but the accuracy of the measurements would be lower. The three-axis accelerometer makes it possible to obtain at any sampling time tₐₖ, the triplet (aₜ_{ₐₖ}^{x}, aₜ_{ₐₖ}^{y}, aₜ_{ₐₖ}^{z}), as well as its modulus defined as ∥A∥ₜ_{ₐₖ}=\sqrt{aₜ_{ₐₖ}^{x}^{²}, aₜ_{ₐₖ}^{y}^{²}, aₜ_{ₐₖ}^{z}^{²}}.

The magnetometer 14 is likewise preferably a magnetometer with three axes so as to be able to directly obtain field measurements on the three axes of the direct trihedron (x, y, z). In accordance with another embodiment, three single-axis magnetometers can be used which are oriented with respect to each other according to the above-mentioned trihedron. The three-axis magnetometer makes it possible to obtain at any sampling time tₘₖ, the triplet (mₜ_{ₘₖ}^{x}, mₜ_{ₘₖ}^{y}, mₜ_{ₘₖ}^{z}), as well as its modulus defined as ∥M∥ₜ_{ₘₖ}=\sqrt{mₜ_{ₘₖ}^{x}^{²}, mₜ_{ₘₖ}^{y}^{²}, mₜ_{ₘₖ}^{z}^{²}}.

The gyroscope 16 is likewise preferably a three-axis gyroscope so as to be able to directly obtain angular velocity measurements on the three axes of the direct trihedron (x, y, z). In accordance with another embodiment, three single-axis gyroscopes can be used which are oriented with respect to each other according to the above-mentioned trihedron. The three-axis gyroscope makes it possible to obtain at any sampling time t_{gk}, the triplet (gₜ_{_{gk}}^{x}, gₜ_{_{gk}}^{y}, gₜ_{_{gk}}^{z}), as well as its modulus defined as ∥G∥ₜ_{_{gk}}={square root over (gₜ_{_{gk}}^{x}^{²}, gₜ_{_{gk}}^{y}^{²}, gₜ_{_{gk}}^{z}^{²})}.

It is also possible to replace the gyroscope and the accelerometer with an inertial measurement unit.

The sampling times tₐₖ, tₘₖ, t_{gk} may be different or the same without compromising the core concept of the invention.

The acquisition of data by the accelerometer 12, magnetometer 14 and gyroscope 16 as well as the other sensors, if applicable, is controlled by a control card housed in the casing 10. This control can, for example, consist of defining the acquisition frequency (the above-mentioned sampling times tₐₖ, tₘₖ, t_{gk}).

The casing 10 also comprises, in accordance with the embodiment of FIG. 2, a radio module 17 which can be used to transmit the data determined and computed by said processing unit to a remote server, in the case where the processing unit is integrated in the casing. When the processing unit is remote, the radio module 17 can be used to initialize the system, verify the presence of the casing, etc.

The casing 10 also comprises, in accordance with the embodiment of FIG. 2, a man-machine interface 18 configured such that the individual can interact with the system and/or a remote operator and receive fall risk notifications. This interface 18 can be of any known type and is not described in detail.

The casing 10 also comprises, in accordance with the embodiment of FIG. 2, a microphone and a loudspeaker 19 which form an audio module configured to permit an exchange of voice information between the individual and a remote operator.

In accordance with one embodiment of the invention, not shown in the figures, the casing 10 can also comprise a pressure sensor and a temperature sensor to derive therefrom information relating to the altitude of the individual wearing the casing.

The system can also comprise, in accordance with an embodiment not shown in the figures, a sphygmomanometer configured to measure the blood pressure of the individual to be monitored and/or a heart rate monitor configured to measure the heart rate of the individual to be monitored.

The processing unit 100 can be integrated in the casing 10 and worn directly by the individual monitored by the system or can be separate and housed on a remote server. In the case where the processing unit 100 is separate, wireless communication means 50 make it possible to connect the sensors housed in the casing 10 and the processing unit 100. These wireless communication means comprise, for example, 3G, 4G, 5G, Wi-Fi connectivity, etc.

This connectivity not only makes it possible to transmit the data from the casing 10 to the processing unit 100, which is for example formed of a software application present on a remote server or a set of software applications and databases present on a set of remote servers (also referred to by the term “cloud”) but also to transmit alerts or information to the casing.

The embodiment described in relation to the figures comprises a processing unit 100 formed of a server remote from the casing. That being said, there is nothing to prevent, in accordance with other embodiments, the processing unit from being partially or completely integrated in the casing worn by the monitored individual.

Therefore, and in accordance with the embodiment of the figures, the processing unit 100 receives the data from the sensors of the casing 10 via wireless communication means 50 connecting the casing 10 and the processing unit 100.

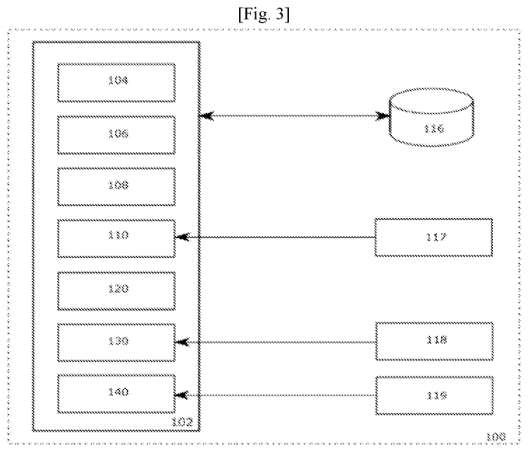

FIG. 3 is a block diagram of the processing unit 100 implemented by a system in accordance with the invention.

The processing unit 100 comprises, for example, a computing device 102 which should be understood in a broad sense (computer, plurality of computers, virtual server on the Internet, virtual server on the cloud, virtual server on a platform, virtual server on a local infrastructure, server networks, etc.). This computing device typically comprises one or more processors 106, one or more memories 108 comprising software routine instructions used by the system and a man-machine interface 104. The processing unit also comprises a database 116 making it possible to save the results of the processing, access information relating to previous processing operations and data relating to the user (which are for example provided via the man-machine interface 104 of the computing device 102 or via the man-machine interface 18 integrated on the casing 10 and wireless communication means 50).

The processing unit 100 comprises four main modules: a module 110 for determining the profile score of the monitored person; a module 120 for computing the motive indices of the monitored person; a module 130 for computing a fall risk score of the monitored person; and a module 140 for determining a risk of falling for the monitored person.

Throughout the rest of this document, the term “module” means a software element, a subset of a software program, which can be compiled separately, either for independent use or to be assembled with other modules of a program, or a hardware element, or a combination of a hardware element and a software subroutine. Such a hardware element can comprise an application-specific integrated circuit (ASIC) or a field-programmable gate array (FPGA) or a digital signal processor (DSP) or any equivalent hardware or any combination of said hardware. Generally, a module is thus an element (software and/or hardware) which makes it possible for a function to be performed.

The processing unit 100 also comprises means for storing the trained computing models 117, 118, 119 implemented by the invention. These means for storing the trained computing models can be servers distinct from the computing device 102 or be saved within the computing device 102.

The automatic computing models implemented by the invention can be of different types. It can be a supervised learning neural network, a support vector machine (SVM) or any other machine learning algorithm.

The different modules of the computing device 102, described hereinafter, use in particular the processors 106, the memories 108, the database 116 and the means for storing the trained computing models 117, 118, 119 in order to be able to be executed.

The module 110 for determining the profile score implements, in accordance with the embodiment of the figures, an automatic computing model 117 trained to determine a profile score of the individual based on the characteristics specific to the individual which are for example saved in the database 116. These specific characteristics comprise for example one or more items of information related to the age, sex, prescribed medications, weight, size, sight, hearing, fall history, etc. of the individual.

The learning database used to train the model is formed of the specific characteristics of a plurality of individuals likely to fall associated with detected fall occurrences for this plurality of individuals.

In order to build such a learning database, a plurality of individuals, whose specific characteristics used by the system (age, sex, weight, etc.) have been noted, were observed (by a dedicated team of operators or by equipping the individuals with a fall detection device) in order to detect fall occurrences. All of the information was saved in a database, forming the learning database of the computing model 117. The training of the model 117 makes it possible for the model to associate a profile score with combinations of characteristics specific to the individuals. In other words, the model 117 makes it possible to determine which are the factors intrinsic to the risk of falling for the individual.

The module 120 for computing the motive indices requests the processor 106 and software routines stored in the memories 108 to evaluate, at predetermined time intervals, the values of the motive indices of the monitored person. The data provided by the sensors are sent to the processing unit 100 by the wireless communication means 50. Of course, and as stated above, it is also possible for provision to be made that the module 120 for computing the motive indices is integrated directly in the casing 10, in which case the motive indices are saved in a memory integrated in the casing 10 and/or transmitted to the remote database 116. A person skilled in the art will easily understand that the location of the different processing operations implemented by the invention is of no importance and can provide different variants without compromising the core concepts of the invention.

In accordance with one embodiment of the invention, the module 120 computes all of the following motive indices:
the indices representative of the average activity of the individual over a predetermined time interval T, referred to as SMA indices, computed from the measurements provided by the accelerometer 12, gyroscope 14 and magnetometer 16 housed in the casing 10 in accordance with the following equations:

$$SM {A}_{A} = \frac{1}{T} ( {\int }_{t}^{t + T} \vert {a}_{t}^{x} \vert dt + {\int }_{t}^{t + T} \vert {a}_{t}^{y} \vert dt + {\int }_{t}^{t + T} \vert {a}_{t}^{z} \vert dt )$$

where aₜ^{x}, aₜ^{y}, aₜ^{z} represent the acceleration values measured on three axes of a direct trihedron (x, y, z) of said accelerometer,

$$SM {A}_{A} = \frac{1}{T} ( {\int }_{t}^{t + T} \vert {m}_{t}^{x} \vert dt + {\int }_{t}^{t + T} \vert {m}_{t}^{y} \vert dt + {\int }_{t}^{t + T} \vert {m}_{t}^{z} \vert dt )$$

where mₜ^{x}, mₜ^{y}, mₜ^{z} represent the magnetic values measured on three axes of said direct trihedron (x, y, z) of the magnetometer,

$$SM {A}_{G} = \frac{1}{T} ( {\int }_{t}^{t + T} \vert {g}_{t}^{x} \vert dt + {\int }_{t}^{t + T} \vert {g}_{t}^{y} \vert dt + {\int }_{t}^{t + T} \vert {g}_{t}^{z} \vert dt )$$

where gₜ^{x}, gₜ^{y}, gₜ^{z} represent the gyroscopic values measured on three axes of said direct trihedron (x, y, z) of the gyroscope,
the indices representative of the current activity of the individual computed from the measurements provided by each of the sensors housed in said casing in accordance with the following equations:

$$\begin{matrix}{‖ A ‖}_{t} = \sqrt{{a}_{t}^{{x}^{2}} + {a}_{t}^{{y}^{2}} + {a}_{t}^{{z}^{2}}} , \\ {‖ M ‖}_{t} = \sqrt{{m}_{t}^{{x}^{2}} + {m}_{t}^{{y}^{2}} + {m}_{t}^{{z}^{2}}} \\ {‖ G ‖}_{t} = \sqrt{{g}_{t}^{{x}^{2}} + {g}_{t}^{{y}^{2}} + {g}_{t}^{{z}^{2}}}\end{matrix}$$

the indices representative of the energy of said individual, referred to as HA indices, computed as the variation in the current activity over said predetermined period T in accordance with the following equations:
   HA_{T}^{A}=var(∥A∥ₜ) where var represents the variation in the value over the predetermined time interval T,
   HA_{T}^{M}=var(∥M∥ₜ) where var represents the variation in the value over the predetermined time interval T,
   HA_{T}^{G}=var(∥G∥ₜ) where var represents the variation in the value over the predetermined time interval T,
the indices representative of the harmony of the activity of the individual, referred to as HM indices, over said predetermined time interval in accordance with the following equations:

$$\begin{matrix}{‖ A ‖}_{t} = \sqrt{{a}_{t}^{{x}^{2}} + {a}_{t}^{{y}^{2}} + {a}_{t}^{{z}^{2}}} , \\ {‖ M ‖}_{t} = \sqrt{{m}_{t}^{{x}^{2}} + {m}_{t}^{{y}^{2}} + {m}_{t}^{{z}^{2}}} \\ {‖ G ‖}_{t} = \sqrt{{g}_{t}^{{x}^{2}} + {g}_{t}^{{y}^{2}} + {g}_{t}^{{z}^{2}}}\end{matrix}$$

the indices representative of the irregularities in the frequency range during the activities of the individual over said predetermined time interval T, referred to as HC indices, measured in accordance with the following equations:

$$\begin{matrix}{HC}_{T}^{A} = \sqrt{\frac{va r ( {‖ A ″ ‖}_{t} )}{va r ( {‖ A ‖}_{t} )}} where {‖ {A}^{″} ‖}_{t} = \frac{Δ ‖ A ′ {‖}_{t}}{Δ t} \\ {HC}_{T}^{M} = \sqrt{\frac{va r ( {‖ M ″ ‖}_{t} )}{va r ( {‖ M ‖}_{t} )}} where {‖ {M}^{″} ‖}_{t} = \frac{Δ ‖ M ′ {‖}_{t}}{Δ t} \\ {HC}_{T}^{G} = \sqrt{\frac{va r ( {‖ G ″ ‖}_{t} )}{va r ( {‖ G ‖}_{t} )}} where {‖ {G}^{″} ‖}_{t} = \frac{Δ ‖ G ′ {‖}_{t}}{Δ t}\end{matrix}$$

These different indices are, for example, saved in the database 116 in order to be able to be used by the other modules of the system.

The module 130 for computing a fall risk score of the monitored individual implements, in accordance with the embodiment of the figures, an automatic computing model 118 trained to compute a fall risk score based on the profile score determined by the module 110 and the motive indices computed by the module 120.

The learning database used to train the model 118 is formed of the motive indices and the profile scores of a plurality of individuals associated with detected fall occurrences for this plurality of individuals.

In order to build such a learning database, a plurality of individuals, whose motive indices have been computed at predetermined time intervals and whose specific characteristics used by the system (age, sex, weight, etc.) have been noted, were observed (by a dedicated team of operators or by equipping the individuals with a fall detection device) in order to detect fall occurrences. All of the information was saved in a database, forming the learning database of the computing model 118. The training of the model 118 makes it possible for this model to associate a fall risk score with combinations of characteristics specific to individuals and motive indices of the individuals. Furthermore, it is possible to regularly enhance the learning database from the users of the system in accordance with the invention (there is collection of the motive indices and characteristics specific to the individuals for whom the system detected a risk of falling, or a fall, confirmed by the individual).

The module 140 for determining a risk of falling implements, in accordance with the embodiment of the figures, an automatic computing model 119 trained to determine a risk of falling from the variations in the fall risk score computed by the module 130, beyond a predetermined threshold.

The learning database used to train the model 119 is formed of the fall risk scores and motive indices of a plurality of individuals associated with detected fall occurrences for this plurality of individuals.

In order to build such a learning database, a plurality of individuals, whose fall risk scores and motive indices have been computed at predetermined time intervals, were observed (by a dedicated team of operators or by equipping the individuals with a fall detection device) in order to detect fall occurrences. All of the information was saved in a database, forming the learning database of the computing model 119. The training of the model 119 makes it possible for the model to associate a risk of falling with sudden variations in the fall risk score.

This makes it possible for the monitored individual to be alerted to the strong probability of a risk of falling within a short amount of time, typically 1 to 5 hours. This alert thus makes it possible to prevent the fall from occurring.

The alert can be transmitted to the individual via the man-machine interface 18 borne by the casing and/or via a voice alert transmitted to the audio module 19 and/or to a person defined by the user of the system as a designated person (a parent, a doctor, etc.) who can then make contact with the monitored individual to alert him/her of a risk of falling and to advise him/her to reduce his/her activity over the next few hours.

A system in accordance with the invention thus makes it possible, by the combination of a set of sensors, different software routines implementing automatic computing models, to predict a risk of falling whereas the earlier solutions were limited to detecting a fall.

The invention also relates to a fall detection system provided with a fall prediction system in accordance with the invention. In particular, the risks of falling provided by the system in accordance with the invention can be used as a parameter of the fall detection system, for example by adapting the detection thresholds to the fall risk information provided by the system.

1. A system for predicting that an individual will fall, comprising:
a casing configured to be able to be worn by an individual, said casing comprising a plurality of sensors for acquiring measurements representative of the posture and/or movements of the individual, including at least one accelerometer, magnetometer and gyroscope,
a unit for processing the measurements provided by said plurality of sensors, said unit comprising
at least:
a module for determining an item of data, referred to as profile score, based on data representative of characteristics specific to the individual,
a module for computing data, referred to as motive indices, based on the measurements provided by said plurality of sensors and representative of the activity of the individual over a predetermined time interval,
a module for computing an item of data, referred to as fall risk score, based on said motive indices and on said profile score,
a module for determining a risk of falling, based on a variation in said fall risk score beyond a predetermined threshold over a predetermined time interval.

2. The system as claimed in claim 1, wherein said module for determining said profile score of said individual comprises an automatic computing model trained to determine a profile score, this computing model, referred to as first model, having been trained using a training database, referred to as profile bank, which comprises data representative of specific characteristics of a plurality of individuals associated with detected fall occurrences for said plurality of individuals.

3. The system as claimed in claim 2, wherein said data representative of the characteristics specific to each individual comprise one or more items of information related to the age, sex, prescribed medications, weight, size, sight, hearing, fall history of said individual.

4. The system as claimed in claim 1, wherein said motive indices computed by said computing module are selected from the group comprising:
indices representative of the average activity of the individual over a predetermined time interval T, referred to as SMA indices, computed from the measurements provided by each of the sensors housed in said casing in accordance with the following equations:
$$SM {A}_{A} = \frac{1}{T} ( {\int }_{t}^{t + T} \vert {a}_{t}^{x} \vert dt + {\int }_{t}^{t + T} \vert {a}_{t}^{y} \vert dt + {\int }_{t}^{t + T} \vert {a}_{t}^{z} \vert dt )$$
where aₜ^{x}, aₜ^{y}, aₜ^{z} represent the acceleration values measured on three axes of a direct trihedron (x, y, z) of said accelerometer,
$$SM {A}_{A} = \frac{1}{T} ( {\int }_{t}^{t + T} \vert {m}_{t}^{x} \vert dt + {\int }_{t}^{t + T} \vert {m}_{t}^{y} \vert dt + {\int }_{t}^{t + T} \vert {m}_{t}^{z} \vert dt )$$
where mₜ^{x}, mₜ^{y}, mₜ^{z} represent the magnetic values measured on three axes of said direct trihedron (x, y, z) of the magnetometer,
$$SM {A}_{G} = \frac{1}{T} ( {\int }_{t}^{t + T} \vert {g}_{t}^{x} \vert dt + {\int }_{t}^{t + T} \vert {g}_{t}^{y} \vert dt + {\int }_{t}^{t + T} \vert {g}_{t}^{z} \vert dt )$$
where gₜ^{x}, gₜ^{y}, gₜ^{z} represent the gyroscopic values measured on three axes of said direct trihedron (x, y, z) of the gyroscope,
indices representative of the current activity of the individual computed from the measurements provided by each of the sensors housed in said casing in accordance with the following equations:
$$\begin{matrix}{‖ A ‖}_{t} = \sqrt{{a}_{t}^{{x}^{2}} + {a}_{t}^{{y}^{2}} + {a}_{t}^{{z}^{2}}} , \\ {‖ M ‖}_{t} = \sqrt{{m}_{t}^{{x}^{2}} + {m}_{t}^{{y}^{2}} + {m}_{t}^{{z}^{2}}} \\ {‖ G ‖}_{t} = \sqrt{{g}_{t}^{{x}^{2}} + {g}_{t}^{{y}^{2}} + {g}_{t}^{{z}^{2}}}\end{matrix}$$
indices representative of the energy of said individual, referred to as HA indices, computed as the variation in the current activity over said predetermined period T in accordance with the following equations:
HA_{T}^{A}=var(∥A∥ₜ) where var represents the variation in the value over the predetermined time interval T,
HA_{T}^{M}=var(∥M∥ₜ) where var represents the variation in the value over the predetermined time interval T,
HA_{T}^{G}=var(∥G∥ₜ) where var represents the variation in the value over the predetermined time interval T,
indices representative of the harmony of the activity of the individual, referred to as HM indices, over said predetermined time interval in accordance with the following equations:
$$\begin{matrix}{HM}_{T}^{A} = \sqrt{\frac{va r ( {‖ A ′ ‖}_{t} )}{va r ( {‖ A ‖}_{t} )}} where {‖ {A}^{′} ‖}_{t} = \frac{Δ ‖ A {‖}_{t}}{Δ t} \\ {HM}_{T}^{M} = \sqrt{\frac{va r ( {‖ M ′ ‖}_{t} )}{va r ( {‖ M ‖}_{t} )}} where {‖ {M}^{′} ‖}_{t} = \frac{Δ ‖ M {‖}_{t}}{Δ t} \\ {HM}_{T}^{G} = \sqrt{\frac{va r ( {‖ G ′ ‖}_{t} )}{va r ( {‖ G ‖}_{t} )}} where {‖ {G}^{′} ‖}_{t} = \frac{Δ ‖ G {‖}_{t}}{Δ t}\end{matrix}$$
indices representative of the irregularities in the frequency range during the activities of the individual over said predetermined time interval T, referred to as HC indices, measured in accordance with the following equations:
$$\begin{matrix}{HC}_{T}^{A} = \sqrt{\frac{va r ( {‖ A ″ ‖}_{t} )}{va r ( {‖ A ‖}_{t} )}} where {‖ {A}^{″} ‖}_{t} = \frac{Δ ‖ A ′ {‖}_{t}}{Δ t} \\ {HC}_{T}^{M} = \sqrt{\frac{va r ( {‖ M ″ ‖}_{t} )}{va r ( {‖ M ‖}_{t} )}} where {‖ {M}^{″} ‖}_{t} = \frac{Δ ‖ M ′ {‖}_{t}}{Δ t} \\ {HC}_{T}^{G} = \sqrt{\frac{va r ( {‖ G ″ ‖}_{t} )}{va r ( {‖ G ‖}_{t} )}} where {‖ {G}^{″} ‖}_{t} = \frac{Δ ‖ G ′ {‖}_{t}}{Δ t}\end{matrix}$$

5. The system as claimed in claim 1, wherein said module for determining said fall risk score comprises an automatic computing model trained to determine a fall risk score, this computing model, referred to as second model, having been trained using a training database, referred to as risk score bank, which comprises values of said motive indices and profile scores of a plurality of individuals associated with detected fall occurrences for said plurality of individuals.

6. The system as claimed in claim 1, wherein said module for determining said risk of falling comprises an automatic computing model trained to determine a risk of falling, this computing model, referred to as third model, having been trained using a training database, referred to as risk bank, which comprises data representative of variations in the fall risk scores of a plurality of individuals associated with detected fall occurrences for said plurality of individuals.

7. The system as claimed in claim 1, wherein it further comprises a radio module configured to be able to transmit the data determined and computed by said processing unit to a remote server.

8. The system as claimed in claim 1, wherein it further comprises an audio module comprising a microphone and a loudspeaker configured to permit an exchange of voice information between the individual and a remote operator.

9. The system as claimed in claim 1, wherein it further comprises a man-machine interface configured such that said individual can interact with said system and/or a remote operator and receive fall risk notifications.

10. A method for predicting that an individual will fall, comprising:
acquiring measurements representative of the posture and/or movement of the individual from at least one accelerometer, magnetometer and gyroscope,
characterized in that it further comprises:
determining an item of data, referred to as profile score, based on data representative of characteristics specific to the individual,
computing data, referred to as motive indices, based on the acquired measurements and representative of the activity of the individual over a predetermined time interval,
computing an item of data, referred to as fall risk score, based on said motive indices and on said profile score,
determining a risk of falling, based on a variation in said fall risk score beyond a predetermined threshold over a predetermined time interval.

11. A system for detecting that an individual has fallen, comprising:
a fall detection module configured to detect that said individual has fallen from at least one measurement from at least one sensor worn by said individual which exceeds a predetermined threshold,
a fall prediction system, configured to determine a fall risk score for said individual,
a module for modifying said predetermined threshold of said fall detection module based on said fall risk score provided by said fall prediction system,
wherein the fall prediction system comprises:
a casing configured to be able to be worn by an individual, said casing comprising a plurality of sensors for acquiring measurements representative of the posture and/or movements of the individual, including at least one accelerometer, magnetometer and gyroscope,
a unit for processing the measurements provided by said plurality of sensors, said unit comprising at least:
a module for determining an item of data, referred to as profile score, based on data representative of characteristics specific to the individual,
a module for computing data, referred to as motive indices, based on the measurements provided by said plurality of sensors and representative of the activity of the individual over a predetermined time interval,
a module for computing an item of data, referred to as fall risk score, based on said motive indices and on said profile score,
a module for determining a risk of falling, based on a variation in said fall risk score beyond a predetermined threshold over a predetermined time interval.
